(19) United States
(12) Patent Application Publication
(10) Pub. No.: US 2009/0292239 A1
(43) Pub. Date: Nov. 26, 2009

(54) Injector System for Needleless, High Pressure Delivery of a Medicament

(75) Inventors: Torben Strom Hansen, Bagsvaerd (DK); Carsten Sorensen, Frederiksberg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/295,427

(22) PCT Filed: Apr. 4, 2007
(86) PCT No.: PCT/EP2007/053311

§ 371 Date: Oct. 28, 2008

(30) Foreign Application Priority Data
Apr. 6, 2006 (EP) 06388026.4

(51) Int. Cl. A61M 5/30

(52) U.S. Cl. 604/72; 604/68

(57) ABSTRACT

An injector system (100) for needleless, high pressure delivery of a medicament, the system comprising: an injector (102) having an injector piston (106) movable with in an injector body (104), a medicament container having a reservoir piston (119) movable in a reservoir (116), a fluid connection (118) interconnecting the injector body and an outlet (120) of the reservoir; and wherein the fluid connection is arranged such, with respect to the injector body, that upon movement of the reservoir piston towards a distal end, the medicament contained in the reservoir is pressurised whereby the injector piston is moved away from an initial position and the injector chamber is filled.

### FIELD OF THE INVENTION

The present invention relates to an injector system for needleless, high pressure delivery of a medicament, the injector system comprising an injector fluidly connected to a medicament container by means of a fluid connection. In particular, the present invention relates to an injector system wherein the fluid connection is arranged such, with respect to the injector, that upon movement of a reservoir piston, a medicament is pressurised whereby an injector piston is moved away from an initial position and an injector chamber is filled.

### BACKGROUND OF THE INVENTION

A large number of patients such as diabetes patients dislike subcutaneous or intramuscular injections due to fear of needles and the associated pain. In order to overcome this problem, needless injection devices have been developed. These devices penetrate the skin using a high velocity liquid jet, whereby the medicament is delivered subcutaneously or intramuscularly without use of needles.

One example of a needleless device is known from U.S. Pat. No. 4,623,332 which discloses a needleless hypodermic injector including cylindrical casing with an injection orifice at a closed front end and a push button at a rear end. A chamber is filled by creating a partial vacuum in the chamber whereby medicine is sucked into the chamber.

Another example is known from WO 01/89613 A1 which discloses an injector device and a method for delivery of liquid from a high pressure, the device comprising a storage chamber and a pressure chamber interconnected by a conduit. The pressure chamber is filled by transferring liquid from the storage chamber to the pressure chamber to only partially fill out the pressure chamber with liquid, and moving a piston forward in the pressure chamber to substantially displace the gas therein through a front end opening of the pressure chamber.

Further background art may be seen in JP 2001-346878, US 2004/030285, WO 2004/-110530, US 2002/055707, WO 2004/032997, US 2001/002433, US 2005/085767, US 2003/036725 and US 2002/007142.

It is an object of a preferred embodiment of the present invention to provide a needleless injection device wherein filling of an injector chamber by vacuum is avoided.

It is an object of a preferred embodiment of the present invention to provide a needleless injection device wherein air in the injector chamber is avoided or limited to a minimum such that preinjection air-evacuation may be avoided or eliminated.

### BRIEF DESCRIPTION OF THE INVENTION

The present invention relates to an injector system for needleless, high pressure delivery of a medicament, the system comprising:
an injector comprising:
   an injector body, and
   an injector piston which is movable within the injector body such that when the injector piston is moved away from an initial position an injector chamber is defined by the injector body and the injector piston, the injector body having a distal end defining an outlet of the injector chamber;
a medicament container comprising:
   a reservoir for accommodation of a medicament, the reservoir defining an outlet in a distal end, and
   a reservoir piston movable within the reservoir;
a fluid connection adapted to be fluidly connected to the outlet of the reservoir;
   wherein the injector body is movable in relation to the fluid connection between:
a first position, wherein the outlet of the fluid connection is not fluidly connected to the outlet of the injector body, and
a second position, wherein the outlet of the fluid connection is fluidly connected to the outlet of the injector body such that when the fluid connection additionally is fluidly connected to the outlet of the reservoir, movement of the reservoir piston towards the distal end causes the medicament to be pressurised whereby the injector piston is moved away from the initial position and the injector chamber is filled; and
   wherein the fluid connection and/or the injector comprises holding means for holding the injector body in each of the first and second position relative to the fluid connection.

In the present invention the injector piston is not retracted prior to filling/loading of the injector chamber in order to effect filling of the injector chamber. On the contrary, the injector is filled by pressurising the reservoir of the medicament container whereby the injector piston is forced away from its initial position and the injector chamber is defined and filled. Accordingly, suction of air is avoided. Moreover, pre-injection air-evacuation is eliminated, which the user experiences as a simplified procedure as fewer steps must be carried out prior to injection.

Upon filling/loading of the injector chamber with a dose of the medicament, the dose may be injected by forcing the piston towards its initial position, whereby the medicament is expelled through the outlet. This may be done manually or by means of an injection mechanism adapted to provide a sufficiently large force to the injector piston to pressurise the medicament so much, that the jet of the medicament expelled through the outlet may penetrate the skin of a living being. In one embodiment the injection mechanism comprises a container with a pressurised gas used to provide the necessary injection force. In another embodiment, the injection mechanism comprises spring which may be strained prior to injection and released during injection so as to force the injector piston towards its initial position. In yet another embodiment, the injection mechanism comprises pyrotechnical means for creating a controlled explosion for forcing the injector piston towards its initial position.

In the context of the present invention the term “initial position” shall be understood as the position wherein the piston cannot be moved any further towards the outlet of the injector and no injector chamber is defined. When the injector piston is positioned in the initial position, at least a part of the distal end of the piston may abut the injector body.

In the context of the present invention, the term “injection” shall be understood as administration of a substance such as a medicament into the skin, subcutaneous tissue, muscle, blood vessels, or body cavities, of a living being.

In the context of the present invention, the term “high pressure delivery of a medicament” shall be understood as delivery of medicaments at a pressure above 100 bar.

The injector body may be substantially cylindrical. Moreover, the cylinder may be designed to be capable of withstanding a high pressure in the injector chamber, such as a pressure above 100 bar, such as above 250 bar.

The outlet of the injector chamber through which the medicament is expelled, may be substantially cylindrical and have a cross-sectional area below 1 mm², such as below 0.5 mm², such as below 0.1 mm², such as below 0.05 mm², such as below 0.03 mm².

The outlet may extend between an inner surface of the injector body and the distal end of the injector body. The length of the outlet may be below 10 mm, such as below 5 mm, such as below 2 mm, such as below 1 mm.

The fluid connection between the injector body and the outlet of the reservoir of the medicament container may be adapted to be changed between an open and a closed position. In the open position a medicament may be transferred from the reservoir to the injector body whereby the injector chamber may be filled. In the closed position fluid cannot be transferred from the injector chamber to the reservoir, whereby pressurization of the medicament during ejection of the medicament does not result in backflow of the medicament from the injection chamber to the reservoir.

In one embodiment, the open and closed position is provided by means of a unidirectional valve preventing flow from the injection chamber to the reservoir. In another embodiment a valve is provided, which valve may be changed manually or automatically between the open and the closed position.

In yet another embodiment, the connection between the injection chamber and the reservoir is adapted to be disconnected after loading of the injection chamber and prior to injection, whereby backflow to the reservoir during injection is avoided.

In order to prevent leakage in the area interconnecting the fluid connection and the injector body, one or more sealing means may be arranged on an outer surface of the injector body and/or the fluid connection such that when the injector body is moved into the second position, the outlet of the injector body is sealingly connected to the outlet of the fluid connection by means of the one or more sealing means. The sealing means may be provided on an outer distal surface of the injector body and/or on a proximal surface of the fluid connection. The sealing means may be provided on surfaces of the injector body and/or the fluid connection which are adapted to abut each other when the injector body is moved into the second position.

In order to facilitate a user with an indication of when the injector body is in the first and/or second position, the holding means may be adapted to provide a tactile and/or auditive and/or visual indication when the injector body is moved into the first and/or second position.

In order to ease handling of the system, especially for users with poor dexterity, the fluid connection may define a fixture for holding the injector in the first and/or second position. In one embodiment the fixture is adapted to maintain the injector body in the first or second position, e.g. by means of a snap lock. In one embodiment the fluid connection defines one or more projection(s) adapted to engage corresponding first or second indentation(s) of the injector body, such that when the projection(s) engage the first indentation(s) the injector body is positioned in the first position and when the projection(s) engage the second indentation(s) the injector body is positioned in the second position. Naturally it will be appreciated, that the indentation(s) may be provided on the fluid connection/fixture and the projection(s) may be provided on the injector body.

In one embodiment the fluid connection defines an outlet terminating on an inner surface of the injector body. Said inner surface may be an abutment surface which the injector piston abuts when the injector piston is in its initial position. In one embodiment the fluid connection defines a plurality of outlets terminating on the inner surface of the injector body.

As an alternative, or as a supplement, the fluid connection may define an outlet terminating on an inner surface of the outlet of the injector body. The diameter of the outlet of the fluid connection may be identical to the diameter of the outlet of the injector body. Alternatively, the diameter of the outlet of the fluid connection may be larger than the diameter of the outlet of the injector body, such as twice the diameter of the injector body, such as three times, such as four times.

As an alternative, or as a supplement, the fluid connection may define an outlet termination on an outer surface of the injector piston such, as a distal facing surface. In this embodiment the fluid connection may be adapted to move with the piston when the injector chamber is filled and/or during expelling of the medicament.

The piston may define a passage for the fluid connection allowing relative movement between the injector piston and the fluid connection e.g. during filling/loading of the injector chamber. In one embodiment the fluid connection comprises a tubular member terminating in an outlet, which, during filling/loading of the injector body, is positioned between a distal surface of the injector piston and the outlet of the injector. Upon pressurising the reservoir the medicament is forced out through the outlet of the fluid connection whereby the injector piston is forced away from its initial position and the injector chamber is filled with the medicament. In one embodiment the fluid connection is adapted to move with the injector piston while in other embodiments the injector piston moves relative to the fluid connection during filling/loading. Prior to injection, the fluid connection is removed. Thus in order to prevent the medicament from leaking through the passage in the injector piston, the injector piston may define a seal for closing the passage when the fluid connection is not inserted into the passage.

Moreover, in order to prevent the medicament from flowing between the fluid connection and the inner surface of the passage during filling/loading of the injector chamber, the passage may comprise a seal adapted to define a seal between an outer surface of the fluid connection and the inner surface of the passage.

In one embodiment, the outlet of the fluid connection may be fluidly connected with the outlet of the injector chamber. As an example the fluid connection may define a surface adapted to abut the distal surface of the injector whereby the outlet of the injector and the outlet of the fluid connection may be aligned when said surfaces abut each other. This allows transfer of the medicament form the reservoir to the injector chamber. In one embodiment the surface the connector and/or the distal surface of the injector comprises alignment means for aligning the outlets.

The alignment means may allow the injector body to move in relation to the fluid connection between: a first position, wherein the outlet of the fluid connection is not fluidly connected to the outlet of the injector body, and a second position, wherein the outlet of the fluid connection is fluidly connected to the outlet of the injector body. This allows evacuation of air from the fluid connection prior to filling/loading of the injector chamber by positioning the injector in the first position and pressurising the reservoir. When a small amount of medicament has emerged on the outer surface of the fluid connection, the injector is moved to the second position whereby transfer of the medicament from the fluid connection to the injector chamber may be effected.

The fluid connection and/or the injector may comprise holding means for holding the injector and the fluid connection in the first and/or second position.

The injector body may comprise sealing means on its distal outer surface for providing a seal between the injector and the fluid connection so as to prevent leakage when a pressurised medicament of the fluid connection is transferred to the injector chamber. Alternatively, or as a supplement the fluid connection may comprise sealing means on its outer surface in the area of its outlet.

### DETAILED DESCRIPTION OF THE INVENTION

In the following the invention is described in further detail with reference to the drawings in which:

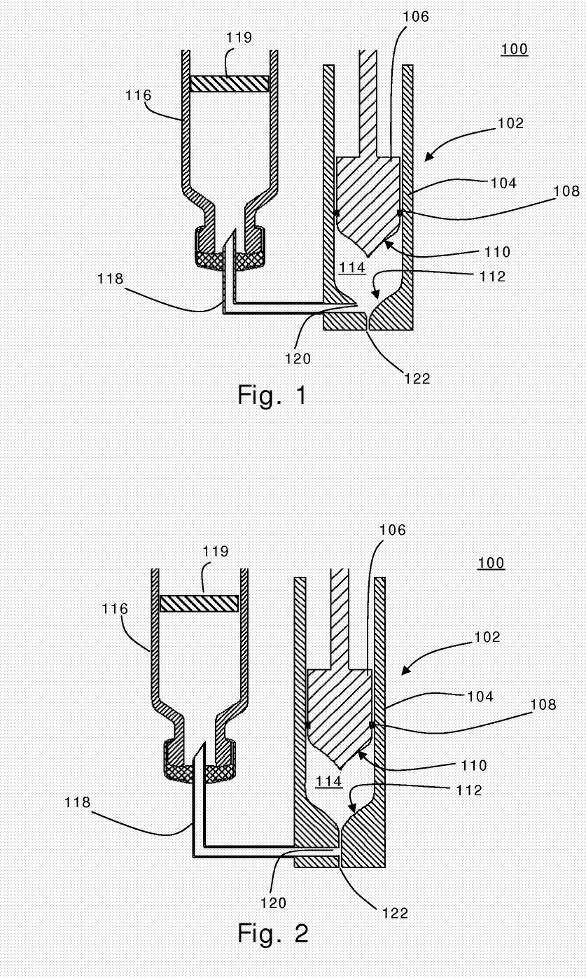

FIG. 1 discloses an injector system, wherein the fluid connection terminates on an inner surface of the injector body,

FIG. 2 discloses an injector system, wherein the fluid connection terminates on an inner surface of the outlet of the injector body,

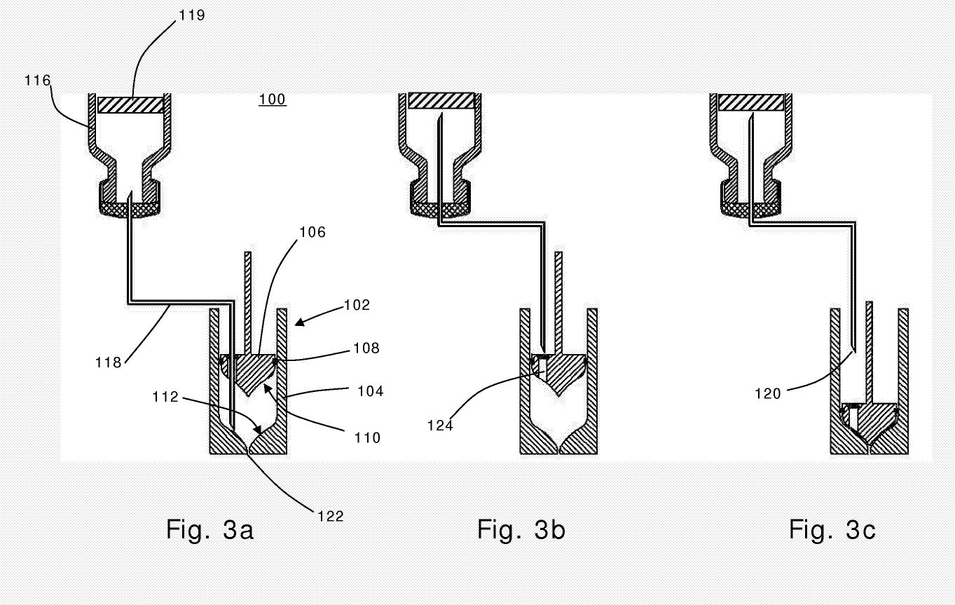

FIGS. 3a-3c disclose an injector system, wherein the injector piston defines a passage for the fluid connection and allowing relative movement between the fluid connection and the piston,

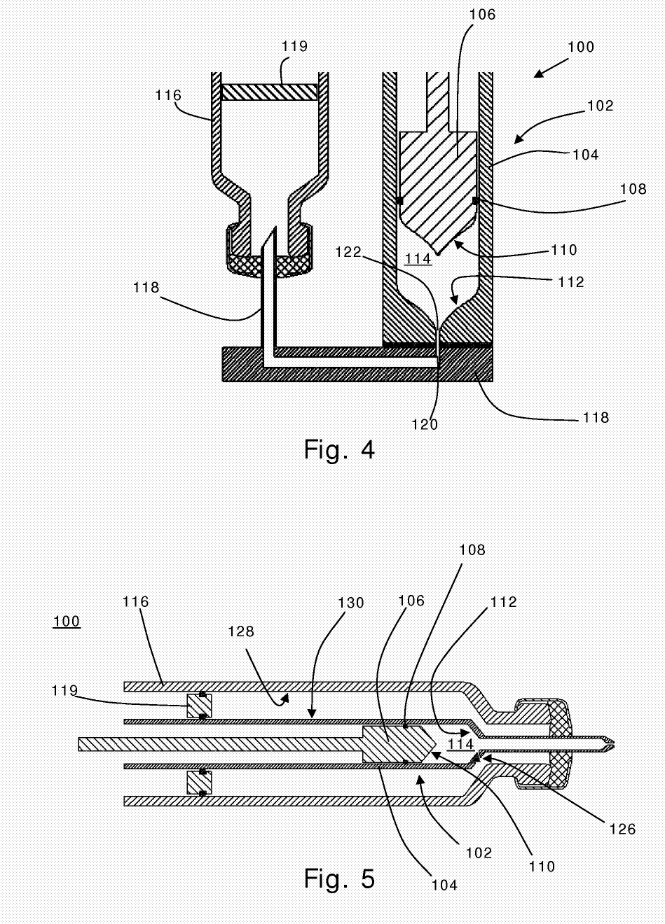

FIG. 4 discloses an injector system, wherein the outlet of the injector body and the fluid connection are adapted to be aligned so as to allow loading of the injector chamber,

FIG. 5 discloses an injector system, wherein the injector in embedded in the medicament container,

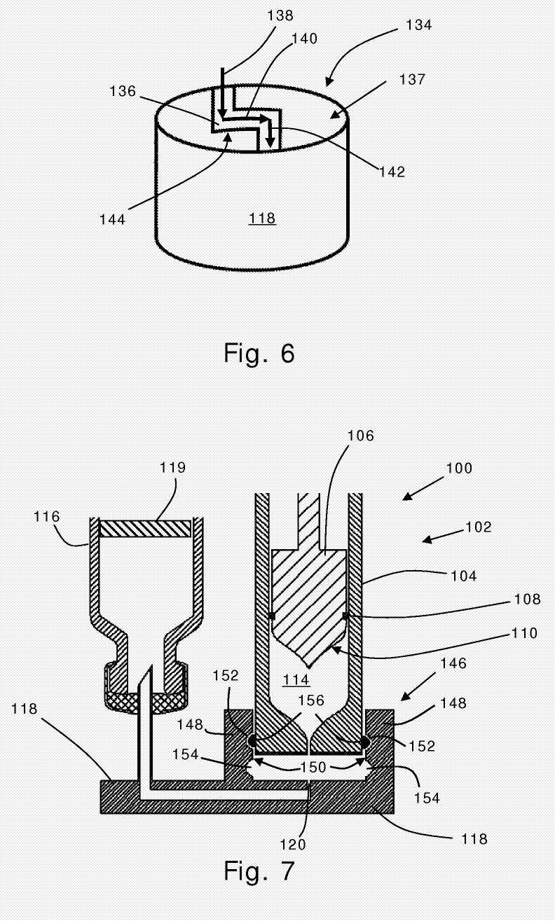

FIG. 6 discloses a first embodiment of means for maintaining the injector body in the first and second position,

FIG. 7 discloses a second embodiment of means for maintaining the injector body in the first and second position, and

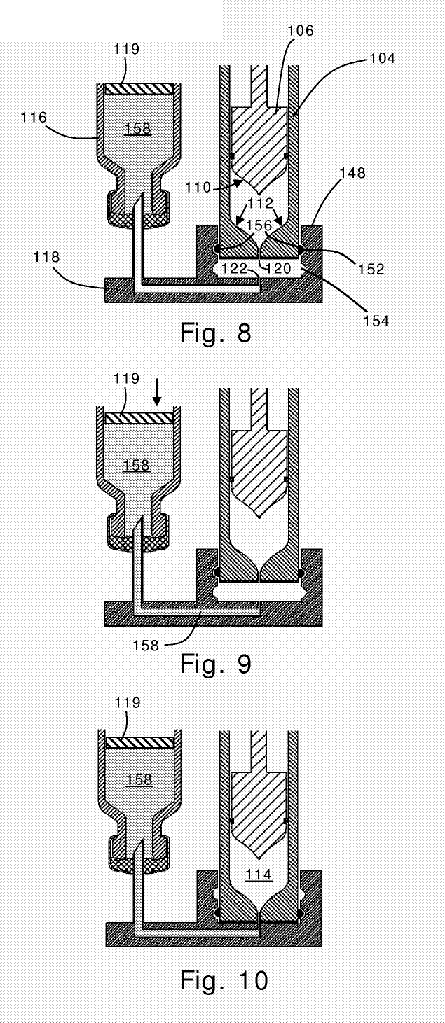

FIGS. 8-10 disclose the process of removing air from the fluid connection.

FIG. 1 discloses an injector system 100 comprising an injector 102 having an injector body 104 and an injector piston 106 movable inside the injector body 104. A seal 108 is provided between the injector body 104 and the injector piston 106. In the drawing the injector piston is moved away from its initial position, in which its distal surface 110 abut a proximal facing surface 112 of the injector body. As the injector piston 106 is moved away from its initial position, an injector chamber 114 is defined by the injector body 104 and the injector piston 106. The injector chamber 114 is fluidly connected to a reservoir 116, by means of a fluid connection 118. A reservoir piston 119 is movable inside the reservoir 116. When the reservoir piston is moved in a distal direction (i.e. downwards in the drawing) a medicament contained in the reservoir is pressurised, and forced through the fluid connection 118, whereby the injector piston 106 is forced away from its initial position and the injector chamber 114 is filled/loaded. The fluid connection comprises an outlet 120 terminating on an inner surface of the injector body 104. The medicament is expelled through the outlet 122 of the injector body 104.

FIG. 2 discloses an injector system 100 comprising identical elements as in FIG. 1 and identical reference numbers refer to identical elements. Relative to FIG. 1 one difference is that the outlet 120 of the fluid connection terminates on an inner surface of the outlet 122 of the injector body.

Again in FIGS. 3a-c identical reference numbers refer to identical elements. One difference relative to FIG. 1 is that the fluid connection extends through a passage of 124 of the injector piston 106. A seal (not shown in FIGS. 3a-c) is provided between the outer surface of the fluid connection 118 and the inner surface of the passage 124. FIG. 3a discloses a filling/loading situation, wherein the injector piston is forced away from its initial position (which is shown in FIG. 3c). In the filling/loading situation the outlet 120 of the fluid connection 118 is positioned in the vicinity of an inner proximal facing surface 122 of the injector body 106. When the injector chamber 114 has been filled with the desired amount of a medicament, the fluid connection 118 is retracted as illustrated in FIG. 3b, and a unidirectional valve (not shown in the drawing) prevents leakage through the passage 124. FIG. 3c illustrates the injector after expelling of the medicament. In this situation the injector piston has returned to its initial position.

In FIG. 4 outlet 120 of the fluid connection 118, is aligned with the outlet 122 of the injector. The injector 102 is adapted to be changed between a first and a second relative position relative to the fluid connection 118. In the first position (not shown in FIG. 4), outlet 120 of the fluid connection 118 is not fluidly connected to the outlet 122 of the injector body 104. In the second position (which is shown in FIG. 4), the outlet 120 of the fluid connection 118 is fluidly connected to the outlet 122 of the injector body 104. The term “fluidly connected” shall be understood such that the two outlets are aligned and positioned in such a way that substantially no leakage in the area of the outlets 120, 122, will occur when a medicament is transferred from the reservoir 116 to the injector 102. In FIG. 4 a seal 127 provided on the distal end of the injector, prevents the leakage.

FIG. 5 discloses an injector system 100 comprising an injector 102 having an injector body 104 and an injector piston 106 movable inside the injector body 104. A seal 108 is provided between the injector body 104 and the injector piston 106. In the drawing the injector piston is moved away from its initial position, in which its distal surface 110 abut a proximal facing surface 112 of the injector body. As the injector piston 106 is moved away from its initial position, an injector chamber 114 is defined by the injector body 104 and the injector piston 106. The injector chamber 114 is fluidly connected to a reservoir 116, by means of a fluid connection 118, defined in the wall of the injector body 104. The fluid connection comprises a unidirectional valve 126 which is adapted to allow a medicament to flow from the reservoir 116 to the injector chamber 114. The reservoir 116 encircles the injector body 104, and the reservoir and the injector body co-extend axially in a longitudinal direction. An outer sidewall of the injector body 104 defines an inner sidewall of the reservoir 116. In one embodiment the centre axis of the reservoir 116 and the injector body 104 coincide. The reservoir piston 118 is movable inside the reservoir 116. The outer lateral surface of the reservoir piston 119 abut an inner surface 128 of the reservoir 116 and the inner surface of the reservoir piston 119 abut an outer surface 130 of the injector body 104. When the reservoir piston 119 is moved in a distal direction (i.e. to the right in the drawing) a medicament contained in the reservoir 116 is pressurised, and forced through the unidirectional valve 132, whereby the injector piston 106 is forced away from its initial position (i.e. in the left direction) and the injector chamber is filled. The unidirectional valve is adapted to prevent flow from the injection chamber 114 to the reservoir 116.

FIG. 6 discloses a first principle for maintaining the injector body in the first and/or second position, wherein the fluid connection 118 defines a cavity 134 for receiving the injector body (not shown). The cavity 134 defines a groove 136 on its inner surface 137, the groove 136 having a first, second and third groove part—indicated by arrows 138, 140, 142. The groove 136 is adapted to receive a corresponding projection (not shown) defined on an outer surface of the injector body 104. Movement of the projection into the first groove part 138, causes the injector body 104 to be moved towards the first position, which is reached when the projection abuts the lower surface 144 of the second groove part 140. In order to move the injector body into the second position, the injector body must be rotated (as indicated by arrow 140) relative to the fluid connection until the lower surface 144 does not prevent further relative axial movement (indicated by arrow 142) between the injector body 118 and the fluid connection 104. Movement of the projection towards the bottom of the third groove part 142, causes the injector body to be moved into the second position. It will be appreciated, that the projections may be provided on the inner surface of the cavity 134, and that the groove 136 may be defined on the outer surface of the injector body 104.

FIG. 7 discloses a second embodiment of means for maintaining the injector body 104 in the first and second position. The fluid connection 118 defines a fixture 146 defining walls 148. On the inner surface 150 of the walls 148 are defined a first set of indentations 152 and a second set of indentations 154. Both sets are adapted to receive the projections 156 of the injector body 104, such that when the projections 156 are positioned in the first set of indentations 152, the injector body 104 is positioned in the first position, and when the projections 156 are positioned in the second set of indentations 154 the injector body 104 is positioned in the second position. Due to the engagement between the projections 156 and the first or second set of indentations 152, 154, the injector body may be maintained in said positions, thus allowing the user to remove air from the conduit of the flow connection 118 as is illustrated in FIGS. 8-10.

FIG. 8 discloses the injector body 104 which has just been positioned in the first position wherein the projections 156 engage the first set of indentations 152 of the walls 148. It will be appreciated that initially the piston 106 will (unlike in the drawing) be positioned in its most distal position wherein the distal surface 110 of the piston 106 abuts the proximal surface 112 injection body 104. Initially the medicament 158 is provided in the reservoir 116 and the cavity of the fluid connection 118 is filled with air. By positioning the injector body 104 in the first position, the outlets 120, 122 are neither sealingly nor fluidly connected and air may be expelled from the fluid connection by moving the piston 119 of the reservoir 116 in the distal direction (downwards in the drawing) which is illustrated in FIG. 9. When air has been evacuated from the cavity of the fluid connection 118, the injector body 104 may be moved into the second position as illustrated in FIG. 10 whereby a sealing member (not shown) provides a seal between the outlets 120, 122. By moving the piston 119 further in the distal direction, the medicament 158 flows into the injector chamber 114.

1. An injector system for needleless, high pressure delivery of a medicament, the system comprising:
an injector comprising:
an injector body, and
an injector piston which is movable within the injector body such that when the injector piston is moved away from an initial position an injector chamber is defined by the injector body and the injector piston, the injector body having a distal end defining an outlet of the injector chamber;
a medicament container comprising:
a reservoir for accommodation of a medicament, the reservoir defining an outlet in a distal end, and
a reservoir piston movable within the reservoir;
a fluid connection adapted to be fluidly connected to the outlet of the reservoir;
wherein the injector body is movable in relation to the fluid connection between:
a first position, wherein the outlet of the fluid connection is not fluidly connected to the outlet of the injector body, and
a second position, wherein the outlet of the fluid connection is fluidly connected to the outlet of the injector body such that when the fluid connection additionally is fluidly connected to the outlet of the reservoir, movement of the reservoir piston towards the distal end causes the medicament to be pressurised whereby the injector piston is moved away from the initial position and the injector chamber is filled; and
wherein the fluid connection and/or the injector comprises holding means for holding the injector body in each of the first and second position relative to the fluid connection.

2. An injector system according to claim 1, wherein one or more sealing means is/are arranged on an outer surface of the injector body and/or the fluid connection such that when the injector body is moved into the second position, the outlet of the injector body is sealingly connected to the outlet of the fluid connection by means of the one or more sealing means.

3. An injector system according to claim 2, wherein the one or more sealing means is provided on an outer distal surface of the injector body.

4. An injector system according to claim 1, wherein the holding means is adapted to provide at least one of a tactile, auditive, or visual indication when the injector body is moved into the first and/or second position.

5. An injector system according to claim 1, wherein the fluid connection defines a fixture for holding the injector in the first and/or second position

6. An injector system according to claim 1, wherein the fluid connection defines an outlet terminating on an inner surface of the injector body.

7. An injector system according to claim 1, wherein the fluid connection defines an outlet terminating on an inner surface of the outlet of the injector body.

8. An injector system according to claim 1, wherein the fluid connection defines an outlet termination on an outer surface of the injector piston.

9. An injector system according to claim 1, wherein the piston defines a passage for the fluid connection allowing relative movement between the injector piston and the fluid connection.

10. An injector system according to claim 9, wherein the passage comprises a seal adapted to define a seal between an outer surface of the fluid connection and the inner surface of the passage.

11. An injector system according to claim 9, wherein the injector piston defines a seal for closing the passage when the fluid connection is not inserted into the passage.

12. An injector system according to claim 1, wherein the outlet of the fluid connection is fluidly connected to the outlet of the injector chamber.

13. An injector system according to claim 1, wherein at least a part of the injector body is encircled by at least a part of the reservoir.

14. An injector system according to claim 10, wherein the injector piston defines a seal for closing the passage when the fluid connection is not inserted into the passage.
